(19) United States
(12) Patent Application Publication
(10) Pub. No.: US 2025/0161612 A1
(43) Pub. Date: May 22, 2025

(54) TRIBOELECTRIC SENSOR FOR IMPROVING FIT OF A PATIENT INTERFACE

(71) Applicant: KONINKLIJKE PHILIPS N.V., EINDHOVEN (NL)

(72) Inventors: DAAN ANTON VAN DEN ENDE, BREDA (NL); PEDRO MIGUEL FERREIRA DOS SANTOS DA FONSECA, BORGERHOUT (BE); JOYCE VAN ZANTEN, EINDHOVEN (NL); CORNELIS PETRUS HENDRIKS, EINDHOVEN (NL); JOACHIM JOHANNES KAHLERT, AACHEN (DE); JONATHAN SAYER GRASHOW, CHESWICK, PA (US)

(21) Appl. No.: 18/949,284

(22) Filed: Nov. 15, 2024

Related U.S. Application Data
(60) Provisional application No. 63/600,116, filed on Nov. 17, 2023.

(51) Int. Cl. A61M 16/06; A61M 16/00

(52) U.S. Cl. CPC A61M 16/0605; A61M 16/0003; A61M 16/024; A61M 2202/0007; A61M 2202/02; A61M 2205/3331; A61M 2205/50; A61M 2210/1025

(57) ABSTRACT

A method of improving fit of a patient interface used in providing a flow of pressurized treatment gas to the airway of a patient includes receiving a signal from a triboelectric sensor provided on the patient interface while the patient interface is being used in providing the flow of the pressurized treatment gas to the airway of the patient. The signal is correlated with flow data of the flow of pressurized treatment gas. From such correlation, at least one change to the patient interface and/or to the flow of pressurized treatment gas is determined. An output based on the at least one change determined is provided.

### CROSS-REFERENCE TO RELATED APPLICATIONS

This patent application claims the priority benefit under 35 U.S.C. § 119 (e) of U.S. Provisional Application No. 63/600,116, filed on Nov. 17, 2023, the contents of which are herein incorporated by reference.

### BACKGROUND OF THE INVENTION

### 1. Field of the Invention

The disclosed concept relates generally to systems and methods for improving fit of patient interfaces used in providing a flow of a pressurized treatment gas to an airway of a patient and, more particularly, to systems and methods which utilize one or more triboelectric sensors in a patient interface to detect movement of one or both of the patient interface and/or the face of the patient during use of the patient interface and use such information to improve fit of the patient interface.

### 2. Description of the Related Art

Today, the first line of therapy for patients diagnosed with obstructive sleep apnea syndrome (OSAS) after a sleep test is a pressure assisted ventilation support, most often by continuous positive airway pressure (CPAP) therapy. In moderate and severe patients with an AHI>15, the therapy is reimbursed by insurance. In mild OSA patients with daytime symptoms, or with chronic and persistent cardiac comorbidities, the PAP therapy is reimbursed for an AHI>5. Reimbursement covers the PAP device as well as periodic resupply of consumable items, such as tubing, headgear, masks and cushions. Depending on geography, different time periods for replacement of these consumable items are in effect, mostly ranging from 1 month to 6 months.

The proper setup of the PAP device, including for instance pressure settings and fitting of the mask, is done by a qualified sleep clinician, most often in an overnight setting at a sleep lab, although home titration is a possible alternative for certain patients. Once the correct machine settings and appropriate consumable items are established, the equipment is supplied by a durable medical equipment (DME) supplier and the patient commences therapy. In the event of difficulties with any aspects of the therapy, a consult, optionally followed by re-titration may be performed, which may result in a change in the machine settings, mask type, or advice on addition/change of comfort features such as humidification settings, use of heated tubes, etc.

Mask leakage is amongst the most frequent causes of discomfort and sub-optimal therapy in OSA patients. This problem can go undetected during (re) titration because the patients sleep is often different in a lab setting than at home, due to different environment, different bedtime habits, interfering equipment etc. In the ideal case, the DME performs a series of routine checkups to monitor the patient's therapy compliance and any issues that may occur during therapy. When a patient is struggling with the mask that was initially prescribed, they may contact their DME or sleep therapist themselves requesting a consultation. Alternatively, the sleep therapist or DME may be alerted by the PAP device's monitoring system if a suspected problem occurs, based on the measured leakage data from the device. Such monitoring systems provide sleep therapists with data from the patient's device measured at home during therapy. Variables that are currently provided include pressure, flow, and leak rate. Currently, arousals and movements are not routinely detected by the PAP devices, although arousals are often linked to respiratory events and (jaw) movements which may affect the mask stability and seal. Better monitoring of sleep details, such as staging, arousals and movements and tracking of vital signs is an ongoing trend in sleep medicine in general, including OSA treatment.

Although the routine follow-ups are good DME practice, especially in the first weeks of therapy, many DMEs do not perform these follow-ups due to time constraints. Typically, patients are offered a different mask in a trial-and-error manner, or they may be redirected to the sleep clinic for a second titration night. In both cases, the effort from both caregivers and patients is significant, without a guarantee for success. The trial-and-error method may have a long throughput time, through which the patient must endure the problems with the therapy, while a re-titration requires the patient to come back to the sleep lab and sleep there for another night. Hence, many patients drop out of therapy instead of contacting their caregivers when experiencing (prolonged) difficulties with their therapy and the risk of dropping out increases when the patient is not served well by the caregiver. This happens often when the caregivers are not fully aware of the root cause of the problems, such as mask problems which could cause leakage or more frequent arousals. In a substantial number of patients, the leakage is not due to a bad initial mask fit, but due to movement of the mask during sleep, which causes it to reposition with respect to the patients face and start leaking during the night, which is important information for caregivers attempting to provide a patient with a new mask to solve their leakage problems. Although the leakage rate can be monitored by the PAP device, there is currently no way to detect a movement or sliding of the mask with respect to the patients face during the night, and hence no way to verify if an increased leak rate is correlated to an arousal and/or mask movement.

### SUMMARY OF THE INVENTION

Embodiments of the disclosed concept address the aforementioned problems and shortcomings of known solutions by utilizing arrangements including triboelectric sensors. As one aspect of the disclosed concept, a method of improving fit of a patient interface used in providing a flow of pressurized treatment gas to the airway of a patient is provided. The method comprises: receiving a signal from a triboelectric sensor provided on the patient interface while the patient interface is being used in providing the flow of the pressurized treatment gas to the airway of the patient; correlating the signal with flow data of the flow of pressurized treatment gas; determining at least one change to the patient interface and/or to the flow of pressurized treatment gas from the correlation of the signal with the flow data; and providing an output based on the at least one change determined.

The signal may be indicative of relative movement between a portion of the patient interface and a corresponding portion of the face of the patient. Determining the at least one change to the patient interface and/or to the flow of pressurized treatment gas may comprise determining a different patient interface and/or a portion thereof that should be employed in providing the flow of the pressurized treatment gas to the airway of the patient. Determining the different patient interface and/or the portion thereof may comprise determining a patient interface or portion thereof that is of a different size. Determining a different patient interface and/or a portion thereof may comprise determining a patient interface or portion thereof that is of a different type. Determining the at least one change to the patient interface and/or to the flow of the pressurized treatment gas may comprise determining at least one adjustment to a fit of a portion of the patient interface. Determining the at least one adjustment to the fit of the portion of the patient interface may comprise determining that the portion should be loosened or tightened on the head of the patient. Determining the at least one change to the patient interface and/or to the flow of pressurized treatment gas may comprise determining an adjustment to a characteristic of the flow of the pressurized treatment gas. Determining the adjustment to the characteristic of the flow of the pressurized treatment gas may comprise determining a new pressure and/or a new flow rate for the flow of the pressurized treatment gas. Providing the output based on the at least one change determined may comprise providing an output that causes the flow of the pressurized treatment gas to the patient to be adjusted to the new pressure and/or the new flow rate. The method may further comprise providing the flow of the pressurized treatment gas to the airway of the patient. Providing the output based on the at least one change determined may comprise providing the output via an output device to one or both of the patient and/or a caregiver of the patient.

As another aspect of the disclosed concept, a system for providing a flow of a pressurized treatment gas to an airway of a patient is provided. The system comprises: a pressure generating device structured to produce the flow of the pressurized treatment gas; a patient interface coupled to, and structured to receive the flow of the pressurized treatment gas from the pressure generating device via a conduit, the patient interface comprising: a body structured to receive the flow of the pressurized treatment gas from the conduit; a sealing member structured to engage with the face of the patient about the airway of the patient; and at least one triboelectric sensor structured to provide a signal indicative of information about one or more of the body and/or the sealing member; and a controller in communication with the at least one triboelectric sensor, the controller structured to: receive a signal from the at least one triboelectric sensor provided on the patient interface while the patient interface is being used in providing the flow of the pressurized treatment gas to the airway of the patient during a therapy session; correlate the signal with flow data of the flow of pressurized treatment gas during the therapy session; determine at least one change to the patient interface and/or to the flow of pressurized treatment gas from the correlation of the signal with the flow data; and provide an output based on the at least one change determined.

The at least one change to the patient interface and/or to the flow of pressurized treatment gas may comprise an adjustment to a characteristic of the flow of the pressurized treatment gas, and the controller may be further structured to cause the pressure generating device to adjust the flow of the pressurized treatment gas provided to the patient in accordance with the adjustment. The at least one triboelectric sensor may comprise a plurality of triboelectric sensors.

These and other objects, features, and characteristics of the disclosed concept, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

A full understanding of the invention can be gained from the following description of the preferred embodiments when read in conjunction with the accompanying drawings in which:

FIGS. 1A-1D are schematic representations of the four main triboelectric

generation modes;

FIG. 2 is partially schematic view of a system in accordance with an example embodiment of the disclosed concept;

FIG. 3 is a flowchart showing steps of a method (along with related events) employing one or more triboelectric sensors positioned on a patient interface in accordance with an example embodiment of the disclosed concept;

FIG. 4A is an example embodiment of a triboelectric sensor arrangement in accordance with an example embodiment of the disclosed concept;

FIG. 4B is a schematic view of the triboelectric sensor components of the arrangement of FIG. 4A; and

FIG. 4C shows schematic representations of the triboelectric sensor components of FIG. 4B positioned in different relative positions with respect to each other in accordance with example embodiments of the disclosed concept.

### DETAILED DESCRIPTION OF THE INVENTION

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

As used herein, the statement that two or more parts or components “engage” one another shall means that the parts exert a force against one another either directly (i.e., “directly engage”) or through one or more intermediate parts or components. As used herein, the word “unitary” means a component is created as a single piece or unit. That is, a component that includes pieces that are created separately and then coupled together as a unit is not a “unitary” component or body. As used herein, the term “number” shall mean one or an integer greater than one (i.e., a plurality).

As used herein, “mask” and “patient interface” are used interchangeably and as such neither of such terms are intended to be more limiting than the other.

As discussed in greater detail below, embodiments of the disclosed concept utilize triboelectric sensor arrangements to detect/monitor movement(s) of a patient interface with respect to a patient and/or movement(s) of the patient's face with respect to the patient interface during a treatment session. Triboelectricity to some extent is present between all material combinations. When two different materials in contact with each other are separated they become electrically charged (static electricity). Reciprocal movement can enhance the (transient) contact between the materials and cause a higher charge buildup in the two separated materials. This charge can be collected by electrodes near the surface of the materials and used as an electric signal for the displacement of the materials. Types of materials can be ranked in a triboelectric series where some materials have affinity for positive charging while others become negatively charged upon separation. The amount of charge that is developed when the materials are separated is increased when the material combination consists of two materials on opposite ends of the spectrum.

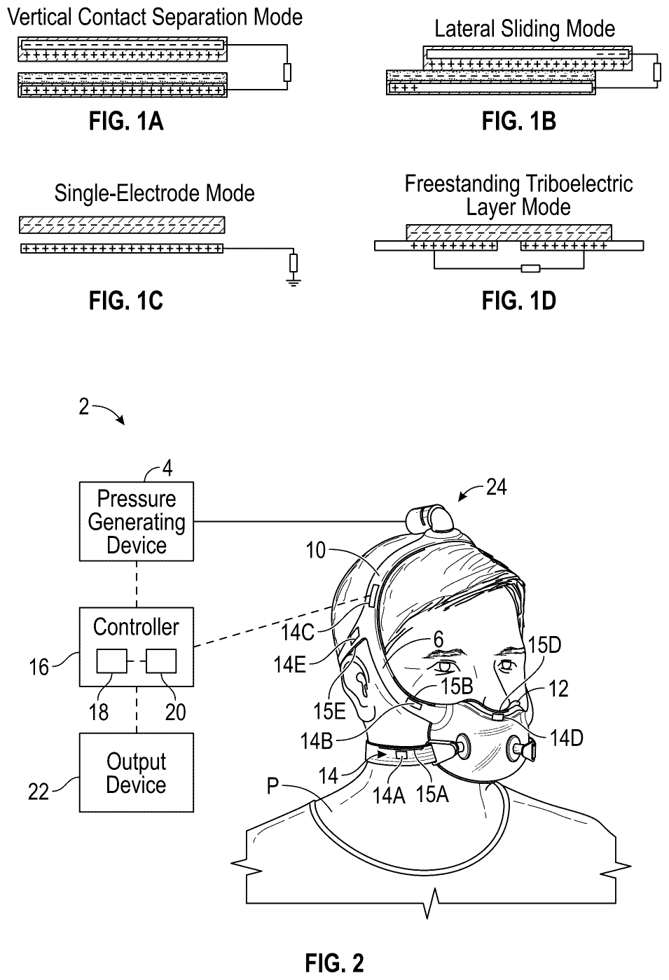

Triboelectric sensors can be made using extremely simple configurations and low-cost materials and are easily compatible with current products and manufacturing methods. Human skin for instance is a highly triboelectric material which is known to be highly affinitive with positive charging while for instance materials such as polyimide, PTFE (Teflon) and silicone rubber predominantly produce negative triboelectric charges. In addition to being simple and low-cost, triboelectric sensors also have the added benefit of being self-powered (i.e., no external power source is needed) and as such the sensors can also be used as micro power generators. Triboelectric sensors have gained popularity in recent years and many examples of triboelectric micro generators or self-powered sensors are known, including systems making use of sheets, films and foams as triboelectric materials and charge transfer mechanisms including contact separation mode and sliding modes or a combination of both. Referring to FIG. 1, there are four main triboelectric generation modes: (A) vertical contact separation mode, (B) lateral sliding mode, (C) single electrode mode and (D) freestanding triboelectric layer mode.

Triboelectric signals are susceptible to variation due to the nature of the signal generation. The exact mechanism of electricity generation is still not fully understood, although it is clear that the nature of the contact, the state of the surface of the material(s) and the environmental conditions all influence the output. Also, triboelectric signals are typically high impedance signals which may be susceptible to noise on the output which must be accommodated for in any design using triboelectricity as a sensor mechanism.

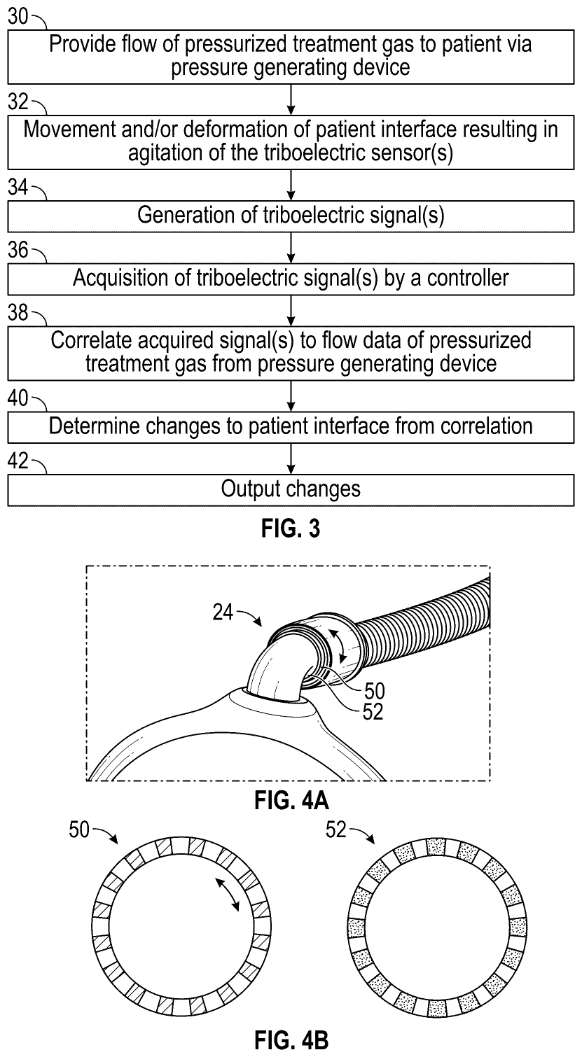

Embodiments of the disclosed concept utilize triboelectric sensor arrangements positioned on a mask/patient interface used in delivering a flow of pressurized treatment gas to the airway of a patient. Depending on the particular application, the triboelectric sensors are positioned so as to generate a signal when the mask is moved and/or deformed. For example, the triboelectric sensor(s) can be positioned so as to produce a signal when movement between the patient interface and patient occurs (e.g., when one or more portion of the patient interface moves during treatment, when one or more portions of the patient's face move with respect to corresponding portions of the patient interface) and/or when movement/deformation/intermittent contact between parts of the masks occurs. A system 2 in accordance with one example embodiment of the disclosed concept is shown in FIG. 2, while a flowchart showing steps of a method employing one or more triboelectric sensors positioned on a patient interface mask along with related events in accordance with an example embodiment of the disclosed concept is shown in FIG. 3.

Referring to FIG. 2, system 2 includes a pressure generating device 4 (shown schematically) coupled to a patient interface 6 via a conduit 8 (also shown schematically). Pressure generating device 4 is structured to produce a flow of a pressurized treatment gas which is communicated to patient interface 6 via conduit 8 for delivery to an airway of a patient P. Patient interface 6 includes a body 10 that is structured to receive the flow of the pressurized treatment gas from conduit 8 and a sealing member 12 structured to engage with the face of the patient about an airway of the patient. While patient interface 6 shown in the example embodiment of FIG. 2 is similar to the Philips Respironics DreamWear Full Face Mask with headgear, it is to be appreciated that such patient interface is provided for exemplary purposes only and that other patient interface arrangements may be employed without varying form the scope of the disclosed concept.

Continuing to refer to FIG. 2, patient interface 6 further includes a number of triboelectric sensors 14 (five are shown schematically, labeled 14A-14E). While the example patient interface 6 shown in FIG. 2 includes five triboelectric sensors 14, each shown located within a respective general region 15A-15E, it is to be appreciated that one or both of the quantity of sensors 14 and/or the location(s) thereof may be varied without varying from the scope of the disclosed concept. It is also to be appreciated that the location(s) in which such sensor(s) may be placed can extend beyond the regions 15 shown. Each triboelectric sensor 14 is in communication (e.g., via any suitable wired or wireless arrangement, discussed further below) with a controller 16 provided as a part of system 2. Controller 16 includes a processing portion 18 which may be, for example, a microprocessor, a microcontroller or some other suitable processing device or devices, and a memory portion 20 that may be internal to processing portion 18 or operatively coupled to processing portion 18 and that provides a storage medium for data and software executable by the processing portion 18. An output device 22 (e.g., a display or any other suitable arrangement may be provided as a part of system 2 for providing output from controller 16. Additionally or alternatively, output from controller 16 may be provided to other arrangements (e.g., without limitation, pressure generating device 4) as discussed further below. Controller 16 may be provided as a portion of pressure generating device 4 controlling functionality thereof or may be a separate element from pressure generating device 4 in communication with a dedicated controller (not numbered) provided as a portion of pressure generating device 4 that controls functionality of pressure generating device 4.

Having thus described a general system in accordance with an example embodiment of the disclosed concept, an example of a method of using such a system 2 and related events will now be described. Referring to the flowchart of FIG. 3, a flow of pressurized treatment gas is provided to patient P via pressure generating device 4, as shown at 30. At any time(s) during such treatment, movement and/or deformation of mask/patient interface 6 and/or movement of the face of patient P occurs that results in agitation of the triboelectric sensor(s) 14 positioned on patient interface 6, such as shown at 32, causing such sensor(s) 14 to generate one or more triboelectric signals, such as shown at 34. As shown at 36, the triboelectric signal(s) generated at 34 is (are) acquired by controller 16 (e.g., via wired or wireless means). At 38, the triboelectric signal(s) acquired at 36 is (are) correlated with flow data of the flow of pressurized treatment gas provided by pressure generating device 4 such that leakages and/or arousals detected by pressure generating device 4 are correlated with movements indicated by the triboelectric sensor(s) 14. From such correlation, one or more changes to the patient interface 6 being utilized by the patient are determined, as shown at 40. Such change(s) may include, for example, without limitation, one or more of the following: a different patient interface 6 should be employed, a different portion of the patient interface 6 should be employed, a portion of the patient interface 6 should be adjusted (e.g., tightened, loosened), a characteristic (e.g., pressure, flow rate, etc.) of the flow of treatment gas provided to the patient should be adjusted. As shown at 42, the change(s) determined at 40 are output. Such output may occur in several different ways. For example, without limitation, such output may be provided via output device as instructions to the patient, caregiver, or any other suitable recipient to carry out the particular changes. As another example, such output may cause such change(s) to be carried out automatically (e.g., cause pressure generating device 4 to change the delivery of the flow, cause an automatic strap adjustment on patient interface to be carried out, etc.).

Having thus described an example system and method in accordance with the disclosed concept some detailed example embodiments will now be described.

### EMBODIMENT 1

### Triboelectric Sensor(s) for Detection of Mask Sliding or Movement and Local Loss of Contact

In this embodiment the triboelectric sensor or sensors are located in the layer that is in contact with the skin. A single sensor can detect sliding of the patient interface with respect to the skin if the sensor slides along the skin or shortly breaks contact via various modes of triboelectricity (e.g., see FIG. 1). This output can be used to identify any movement or repositioning of the patient interface. The duration and magnitude of the output can be used to estimate the extent of the movement. By time synchronizing this data with any change in leakage that is recorded by the PAP device (e.g., pressure generator 4), these events can be correlated to any changes in leak of the patient interface that may occur during the night. Such information can be used by sleep physicians to evaluate the cause of changes in leakage, for instance abrupt changes may indicate tossing and turning whereas gradual movement may indicate a patient interface slowly slipping from the correct position, for instance due to the strapping force being too low or a poor fit.

More information can be gained by an array of sensors in the seal portion of the patient interface. When the patient interface slides during the night and/or the seal is broken locally, the sensors in the array where the seal is broken will give a larger output signal than the others, caused by the vertical contact separation mode of the triboelectric sensor (see FIG. 1). The localization of the breaking point of the seal may allow better insight into the causes for poor sealing occurring during sleep, especially if the seal breaks repeatedly at the same location. In this way, it may help physicians or DMEs solve mask problems quickly and remotely, such as giving targeted mask positioning advice or advice to tighten or loosen top or bottom straps only. Optionally, the CPAP device automatically adjusts the CPAP pressure after registering a mask movement in combination with a change in unintentional leakage, in order to optimize therapy delivery and/or comfort.

Optionally, a sensor arrangement may be employed using an array of triboelectric sensors where each sensor element differs in rectangular and/or elliptic shape. These basic sensor elements are arranged in orthogonal orientation. By a voltage and charge analysis of all basic sensor elements, a movement vector can be calculated to determine the orientation of the movement. The sensors in the seal can typically be made of conductive rubber areas (e.g., silicone with a conductive additive) to act as electrodes or wires such that the sensors are truly embedded in the seal.

### EMBODIMENT 2

### A Triboelectric Sensor in Combination with an Accelerometer for Accurate Detection of Mask Sliding with Respect to Head Movement

This embodiment employs a triboelectric sensor and a second triboelectric sensor detecting position of the head, or other means of determining the head position of the patient during sleep such as an accelerometer. The movement data from the triboelectric sensor or sensor array on the cushion will, in combination with the head position or change in head position at the time of the registered mask movement, give accurate insight into the type of movement and/or head position at the time of movement that causes slipping of the mask and/or breaking of the seal at a certain position.

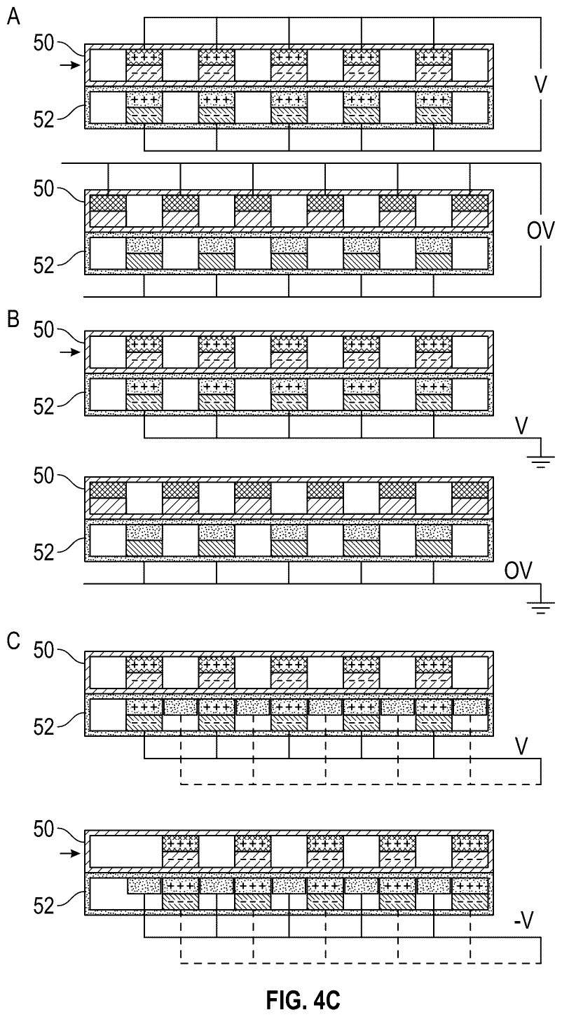

A triboelectric sensor that detects head position change can for instance be located in a swivel connection 24, such as shown in FIGS. 2 and 4A. For instance, two rings 50 and 52, located on mating surfaces in hose swivel connection 24 may be equipped with alternating patterns of triboelectric materials consisting of a triboelectric series, as shown in FIG. 4B. The filler material among such patterns is a neutral material. When swivel connection 24 turns, the opposite contact sides rotate with respect to each other and a triboelectric signal is generated.

For instance, in one configuration A as shown in FIG. 4C, one contact side is free to rotate (e.g., ring 50 in FIG. 4B), while the other part (e.g., ring 52 in FIG. 4B) is fixed. Each rotation results in a signal generation depending on the precise combination of materials and electrode configuration. Different setups, such as shown in A, B and C in FIG. 4C can be optimized for signal output. The output of such arrangement, in combination with the output of one or more skin-facing triboelectric sensors can correlate head movement with slipping of the patient interface. Alternatively, head position of the patient may be sensed by an accelerometer. This has the advantage that the absolute orientation of the head can be measured, instead of only movements. This way the orientation of the patient's head may be correlated to the occurrence of mask slipping or leakage. The electricity generated by the triboelectric element in FIG. 4A can be used to power an accelerometer that determines head position, or any other sensor that is located in the patient interface. It may also be used to power other electronics such as wireless data transfer electronics and/or recharge a power storage element such as a battery in the patient interface. The localization of the breaking point of the seal in combination with the head position can provide better insight into the causes for poor mask seal occurring during sleep, especially if the seal breaks repeatedly at the same location or head position/orientation. It thus may help physicians or DMEs solve mask problems quickly and remotely, for instance advising to tighten the straps on one side a little more than the other side to avoid mask leakage when the patient is lying on their (preferred) side.

### EMBODIMENT 3

### Detection of Unwanted Mask Deformation During Sleep

In this embodiment a triboelectric sensor is embedded in a certain area of the mask, such that unwanted deformation of that part of the mask can be detected. For instance, a triboelectric sensor embedded in one of the tubes of a Philips Respironics DreamWear mask that run along the side of the face will be able to detect when this tube is accidentally crushed (i.e. when the patient lays on their side). With today's technology the PAP device cannot correct for the pressure drop change when a patient crushes one of the tubes because it cannot detect when it happens. The triboelectric contact mode measurement may be used to detect when the two opposing inner surfaces of the tube come into contact, when the tube is crushed and collapses. Such a signal will typically be very reproducible due to specific collapse kinetics of the tube, which are governed by geometry and materials used. When the triboelectric sensor signals such an obstruction the air pressure may be temporarily adjusted accordingly until the triboelectric sensor detects that the tube surfaces have released.

### EMBODIMENT 4

### A Triboelectric Sensor for Detecting Muscle Movements Associated with Arousals and Mask Sliding Movements

In this embodiment a triboelectric sensor is positioned in the headgear where the headgear straps pass over the jaw closing masseter muscle (MAS) or in the mask cushion near the chin. As an example, MAS activity is associated with arousals, the amount of muscle activity progresses with arousal severity. Triboelectric sensors based on roughened PET and PTFE combination that are capable of measuring slight muscle movements have been described in literature. These sensors have been proven to detect jaw muscle movement, eye blinks and even heart rate (pulse). In this embodiment the controller is configured to detect MAS activation from the specific response of the sensor (i.e., with a certain specific voltage profile) to distinguish it from general sliding or movement of the mask (i.e., ‘other’ signals).

### EMBODIMENT 5

### A Triboelectric Sensor Array for Detecting Multiple Muscle Activations Associated with Severity of Arousals

In this embodiment several triboelectric sensors are embedded in the mask at the locations of at least two different muscles (for instance both jaw and chin muscles).

The level/intensity of arousal (e.g., from micro-arousal up to an awakening) is determined from the number of locations it “activates”, i.e., from the number and/or intensity of the different triboelectric sensor signals. It is known from literature that: “The number of muscles activated in response to a single spontaneous arousal was higher for AW [awakenings] (1.6 [1.3-2.0]) than for mAR [arousals] (0.8 [0-2.0]) (Wilcoxon test: p<0.001).”Thus, longer (or more intense) arousals activate more muscles, and this can be seen by using multiple triboelectric sensors at different locations on the face.

### EMBODIMENT 6

### Mouth Opening Detection

In another embodiment, a configuration of sensors is employed to measure mouth opening and closing. Jaw opening is known to decrease at the termination of apneic episodes. In this embodiment, one sensor detects the arousal (e.g., using the increased muscle activation as in embodiment 4) and another sensor detects mouth closing upon termination of the apneic event. If both signals are present this would indicate whether that the arousal is related to a residual SDB problem (e.g., one not properly addressed by the set CPAP pressure, or one where the auto-PAP machine failed to adjust to RERAs, or events during REM which are known to be difficult to always address) which may require an adjustment of the therapy, or to other causes which deserve further investigation if the patient is presenting symptoms (e.g., residual daytime sleepiness etc.).

### EMBODIMENT 7

### Sleep Bruxism Detection

In this embodiment the whole night total jaw motor activity is monitored. Patients that experience sleep bruxism are known to exhibit a significantly larger total activity duration than patients without sleep bruxism. The total activity time and intensity as registered by the triboelectric sensor(s) is analyzed by the controller to determine if the patient is suspected to suffer from sleep bruxism, for instance if the total jaw motor activity exceeds a set threshold.

From the foregoing, it is thus to be appreciated that embodiments of the disclosed concept provide arrangements for improving delivery of a CPAP treatment to a patient by monitoring multiple variables during treatment and finding correlations therefrom that can be used to improve further treatment sessions by improving the fit of a patient interface or portion(s) thereof employed during such sessions.

While specific embodiments of the disclosed concept have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of disclosed concept which is to be given the full breadth of the claims appended and any and all equivalents thereof.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

What is claimed is:

1. A method of improving fit of a patient interface used in providing a flow of pressurized treatment gas to the airway of a patient, the method comprising:
receiving a signal from a triboelectric sensor provided on the patient interface while the patient interface is being used in providing the flow of the pressurized treatment gas to the airway of the patient;
correlating the signal with flow data of the flow of pressurized treatment gas;
determining at least one change to the patient interface and/or to the flow of pressurized treatment gas from the correlation of the signal with the flow data; and
providing an output based on the at least one change determined.

2. The method of claim 1, wherein the signal is indicative of relative movement between a portion of the patient interface and a corresponding portion of the face of the patient.

3. The method of claim 1, wherein determining the at least one change to the patient interface and/or to the flow of pressurized treatment gas comprises determining a different patient interface and/or a portion thereof that should be employed in providing the flow of the pressurized treatment gas to the airway of the patient.

4. The method of claim 3, wherein determining the different patient interface and/or the portion thereof comprises determining a patient interface or portion thereof that is of a different size.

5. The method of claim 3, wherein determining the different patient interface and/or the portion thereof comprises determining a patient interface or portion thereof that is of a different type.

6. The method of claim 1, wherein determining the at least one change to the patient interface and/or to the flow of pressurized treatment gas comprises determining at least one adjustment to a fit of a portion of the patient interface.

7. The method of claim 6, wherein determining the at least one adjustment to the fit of the portion of the patient interface comprises determining that the portion should be loosened or tightened on the head of the patient.

8. The method of claim 1, wherein determining the at least one change to the patient interface and/or to the flow of pressurized treatment gas comprises determining an adjustment to a characteristic of the flow of the pressurized treatment gas.

9. The method of claim 8, wherein determining the adjustment to the characteristic of the flow of the pressurized treatment gas comprises determining a new pressure and/or a new flow rate for the flow of the pressurized treatment gas.

10. The method of claim 9, wherein providing the output based on the at least one change determined comprises providing an output that causes the flow of the pressurized treatment gas to the patient to be adjusted to the new pressure and/or the new flow rate.

11. The method of claim 1, further comprising providing the flow of the pressurized treatment gas to the airway of the patient.

12. The method of claim 1, wherein providing the output based on the at least one change determined comprises providing the output via an output device to one or both of the patient and/or a caregiver of the patient.

13. A system for providing a flow of a pressurized treatment gas to an airway of a patient, the system comprising:
a pressure generating device structured to produce the flow of the pressurized treatment gas;
a patient interface coupled to, and structured to receive the flow of the pressurized treatment gas from the pressure generating device via a conduit, the patient interface comprising:
a body structured to receive the flow of the pressurized treatment gas from the conduit;
a sealing member structured to engage with the face of the patient about the airway of the patient; and
at least one triboelectric sensor structured to provide a signal indicative of information about one or more of the body and/or the sealing member; and
a controller in communication with the at least one triboelectric sensor, the controller structured to:
receive a signal from the at least one triboelectric sensor provided on the patient interface while the patient interface is being used in providing the flow of the pressurized treatment gas to the airway of the patient during a therapy session;
correlate the signal with flow data of the flow of pressurized treatment gas during the therapy session;
determine at least one change to the patient interface and/or to the flow of pressurized treatment gas from the correlation of the signal with the flow data; and
provide an output based on the at least one change determined.

14. The system of claim 13, wherein the at least one change to the patient interface and/or to the flow of pressurized treatment gas comprises an adjustment to a characteristic of the flow of the pressurized treatment gas, and wherein the controller is further structured to cause the pressure generating device to adjust the flow of the pressurized treatment gas provided to the patient in accordance with the adjustment.

15. The system of claim 13, wherein the at least one triboelectric sensor comprises a plurality of triboelectric sensors.
